(19) United States
(12) Patent Application Publication
(10) Pub. No.: US 2016/0078778 A1
(43) Pub. Date: Mar. 17, 2016

(54) ONLINE BEHAVIORAL AND PHYSICAL HEALTH MONITORING SYSTEM

(71) Applicant: Matthew Ryan Holland, Salem, OR (US)

(72) Inventor: Matthew Ryan Holland, Salem, OR (US)

(21) Appl. No.: 14/688,712

(22) Filed: Apr. 16, 2015

Related U.S. Application Data
(60) Provisional application No. 62/049,248, filed on Sep. 11, 2014.

(51) Int. Cl. G09B 19/00; A61B 5/00; G09B 5/02

(52) U.S. Cl. CPC G09B 19/00; G09B 5/02; A61B 5/746

(57) ABSTRACT

A system for monitoring health may track end user physical and behavioral wellness factors. The factors may be input daily. Requests for input may be prompted by trends or triggers in the end user's health profile. Medical intervention may be prompted by evaluating data or end user feedback.

### CROSS-REFERENCE TO RELATED APPLICATIONS

This application claims benefit under 35 U.S.C. §119(e) of U.S. Provisional Application Ser. No. 62/049,248 filed Sep. 11, 2014, which is hereby incorporated by reference herein in its entirety.

### BACKGROUND

The embodiments herein relate generally to systems providing online monitoring of wellness.

In the world of behavioral and physical health people often are unable to get daily care and connection from their providers. Funders, providers and consumers typically track wellness only on scheduled visits and at extended frequencies. Conventional devices or systems currently in use do not track daily wellness for the ability of patient centered collaboration. Nor systems currently have the ability to pull daily stored data for review by funders, providers or consumers. The result is a lack in proper funding and treatment for approaches to treat the individual. The disclosed invention solves these problems by providing a secure electronic online alert and/or modular system featuring behavioral health and physical health technology which allows the users to collaboratively upload information that is tracked and stored daily in a database through a mobile app. This information is then configured into graphs and charts for later review by one or more users (funders, providers and consumers).

### SUMMARY

A computer program product for monitoring behavioral wellness of a user, the computer program product comprising a non-transitory computer readable storage medium having computer readable program code embodied therewith, the computer readable program code being configured to: provide a portal, accessed through a computing device, for user input of behavioral wellness data; prompt an end user on a display of the computing device for daily input of behavioral wellness data; generate, via a processor, real-time wellness reports based on the input of behavioral wellness data to a provider; and trigger an alert for intervention to a provider based on the real-time wellness reports indicating criteria being met requiring a need for assistance to the user.

### BRIEF DESCRIPTION OF THE FIGURES

The detailed description of some embodiments of the invention is made below with reference to the accompanying figures, wherein like numerals represent corresponding parts of the figures.

FIG. 1 is a block diagram of a computer system/server according to an embodiment of the subject technology.

FIG. 2 is a block diagram of a network according to an embodiment of the subject technology.

FIG. 3A is an exemplary user interface module dashboard shown in use on a mobile device according to an embodiment of the subject technology.

FIG. 3B is an exemplary module interface displayed in response to selecting a module in FIG. 3A according to an embodiment of the subject technology.

FIG. 3C is an exemplary module interface displayed in response to selecting a question in the module interface of FIG. 3B according to an embodiment of the subject technology.

FIG. 3D is another exemplary module interface displayed in response to selecting another question in the module interface of FIG. 3B according to an embodiment of the subject technology.

FIG. 4A is another exemplary user interface module dashboard shown in use on a mobile device according to an embodiment of the subject technology.

FIG. 4B is another exemplary module interface displayed in response to selecting a module in FIG. 4A according to an embodiment of the subject technology.

FIG. 5 is a screenshot of an exemplary client manager user interface according to an embodiment of the subject technology.

FIG. 6 is a chart of exemplary modules used according to embodiments of the subject technology.

FIG. 7A is a screenshot of an exemplary coping skills module according to an embodiment of the subject technology.

FIG. 7B is a screenshot of a system triggered module showing a list of recommended activities based on user provided feedback from the survey of FIG. 7A.

FIG. 8 is a screenshot of an exemplary distress module according to an embodiment of the subject technology.

FIG. 9 is a screenshot of an exemplary crisis module according to an embodiment of the subject technology.

FIG. 10 is a flow diagram of a method of triggering an alert according to an embodiment of the subject technology.

### DETAILED DESCRIPTION OF CERTAIN EMBODIMENTS

In general, embodiments of the disclosed invention provide an online module system for providing end users interaction with a network that provides the end user with surveys/questions that may prompt an automatic response based on the user's feedback. In an exemplary embodiment, a portal to the online modules and feedback of the system may be used for daily monitoring of an individual's wellness for better diagnostic results and the issuance of alerts to intervene before a client's health becomes dire. Embodiments provide means to look at data to preemptively target warning signs of health risks which may fund better approaches to treatment. An exemplary embodiment may be provided online by connecting an end user to a system tracking the end user's physical and behavioral wellness. Input from the end user may be processed and the results can be evaluated by health professionals to determine, for example, whether some form of intervention may be needed before the end user's wellness deteriorates into a dangerous situation.

In the description below, referring to behavioral wellness may be related to, for example a state of mental/emotional being and/or choices and actions that affect a person's wellness. Substance abuse and misuse are one example set of behavioral health/wellness problems. Other behavioral health problems include (but are not limited to) serious psychological distress, suicide, and mental illness. Such problems are far-reaching and exact an enormous toll on individuals, their families and communities, and the broader society.

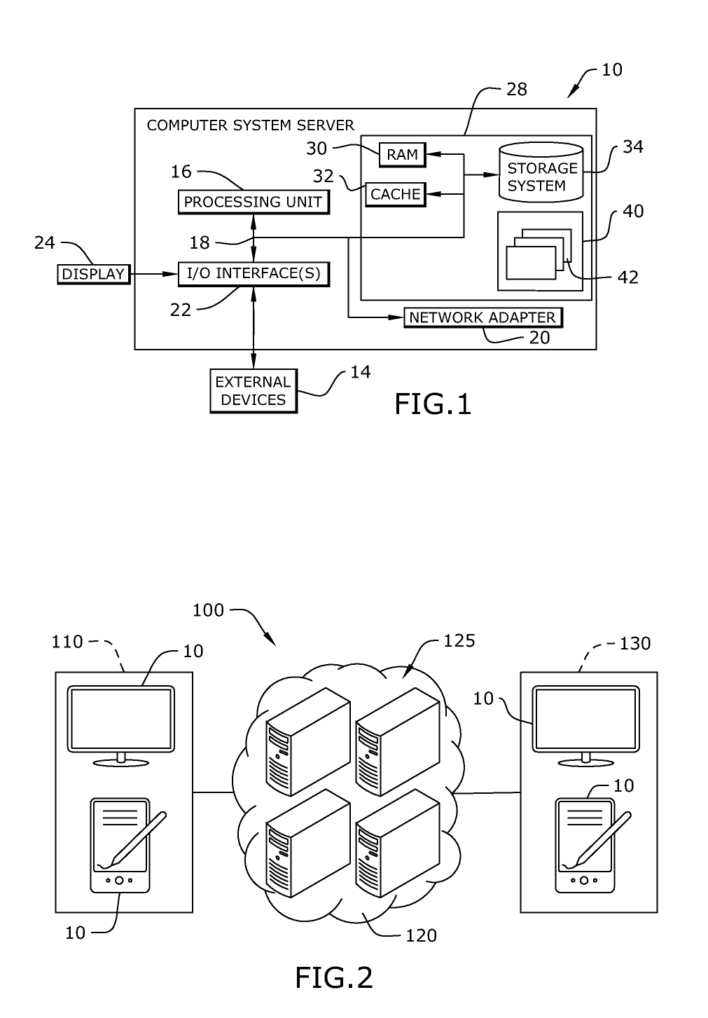

Referring now to FIG. 1, a schematic of an example of a computer system/server 10 is shown. The computer system/server 10 is shown in the form of a general-purpose computing device. The computer system/server 10 may serve the role as the machine implementing for example the functions of storing client records, providing a user interface platform, providing a web portal for access by end client users, physicians, and third parties, sending out modules to users in the system, receiving user input/feedback, invoking follow-up modules in response to user input/feedback, storing historical records of user data input/feedback, providing user data to third parties, providing user initiated requests to third parties, determining user behavior needing attention, and triggering automatic actions/alerts. The components of the computer system/server 10 may include, but are not limited to, one or more processors or processing units 16, a system memory 28, and a bus 18 that couples various system components including the system memory 28 to the processor 16.

The computer system/server 10 may perform functions as different machine types depending on the role in the system the function is related to. For example, depending on the function being implemented at any given time when interfacing with the system, the computer system/server 10 may be for example, personal computer systems, tablet devices, mobile telephone devices, server computer systems, handheld or laptop devices, multiprocessor systems, microprocessor-based systems, set top boxes, programmable consumer electronics, network PCs, and distributed cloud computing environments that include any of the above systems or devices, and the like. The computer system/server 10 may be described in the general context of computer system executable instructions, such as program modules, being executed by a computer system (described for example, below). In some embodiments, the computer system/server 10 may be a cloud computing node connected to a cloud computing network (not shown). The computer system/server 10 may be practiced in distributed cloud computing environments where tasks are performed by remote processing devices that are linked through a communications network. In a distributed cloud computing environment, program modules may be located in both local and remote computer system storage media including memory storage devices.

The computer system/server 10 may typically include a variety of computer system readable media. Such media could be chosen from any available media that is accessible by the computer system/server 10, including non-transitory, volatile and non-volatile media, removable and non-removable media. The system memory 28 could include one or more computer system readable media in the form of volatile memory, such as a random access memory (RAM) 30 and/or a cache memory 32. By way of example only, a storage system 34 can be provided for reading from and writing to a non-removable, non-volatile magnetic media device typically called a “hard drive” (not shown). The system memory 28 may include at least one program product 40 having a set (e.g., at least one) of program modules 42 that are configured to carry out the functions of embodiments of the invention. The program product/utility 40, having a set (at least one) of program modules 42, may be stored in the system memory 28 by way of example, and not limitation, as well as an operating system, one or more application programs, other program modules, and program data. Each of the operating system, one or more application programs, other program modules, and program data or some combination thereof, may include an implementation of a networking environment. The program modules 42 generally carry out the functions and/or methodologies of embodiments of the invention as described herein.

The computer system/server 10 may also communicate with one or more external devices 14 such as a keyboard, a pointing device, a display 24, etc.; and/or any devices (e.g., network card, modem, etc.) that enable the computer system/server 10 to communicate with one or more other computing devices. Such communication can occur via Input/Output (I/O) interfaces 22. Alternatively, the computer system/server 10 can communicate with one or more networks such as a local area network (LAN), a general wide area network (WAN), and/or a public network (e.g., the Internet) via a network adapter 20. As depicted, the network adapter 20 may communicate with the other components of the computer system/server 10 via the bus 18.

As will be appreciated by one skilled in the art, aspects of the disclosed invention may be embodied as a system, method or process, or computer program product. Accordingly, aspects of the disclosed invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the disclosed invention may take the form of a computer program product embodied in one or more computer readable media having computer readable program code embodied thereon.

Any combination of one or more computer readable media (for example, storage system 34) may be utilized. In the context of this disclosure, a computer readable storage medium may be any tangible or non-transitory medium that can contain, or store a program (for example, the program product 40) for use by or in connection with an instruction execution system, apparatus, or device. A computer readable storage medium may be, for example, but not limited to, an electronic, magnetic, optical, electromagnetic, infrared, or semiconductor system, apparatus, or device, or any suitable combination of the foregoing.

Aspects of the disclosed invention are described below with reference to block diagrams of methods, apparatus (systems) and computer program products according to embodiments of the invention. It will be understood that each block of the block diagrams, and combinations of blocks in the flowchart illustrations and/or block diagrams, can be implemented by computer program instructions. These computer program instructions may be provided to the processor 16 of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

Referring now to FIG. 2, a block diagram of a system 100 for monitoring the wellness of an individual is shown. The system 100 may connect an end user 110 to a provider 130 through a network 120. The network 120 may include a server 125 storing a software embodiment of the disclosed invention. The end user 110 and provider 130 may interact with the system 100 with an electronic device (for example, a PC or mobile device). It will be understood that the electronic device used by the end user and the provider and the server 125 may function for example, under the description the computer system/server 10 of FIG. 1. In some embodiments, the network 120 may be a cloud based environment. In some embodiments, client records related to their physical and behavioral wellness may be stored centralized in the server 125 for access by either the provider or end user client.

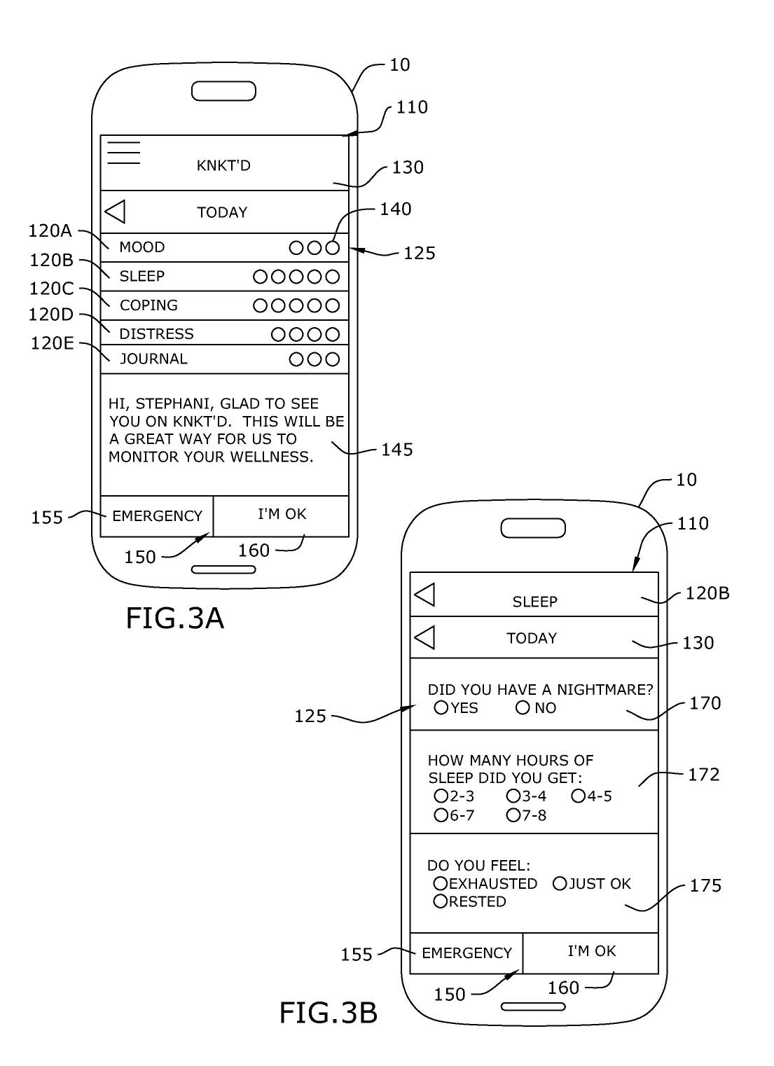

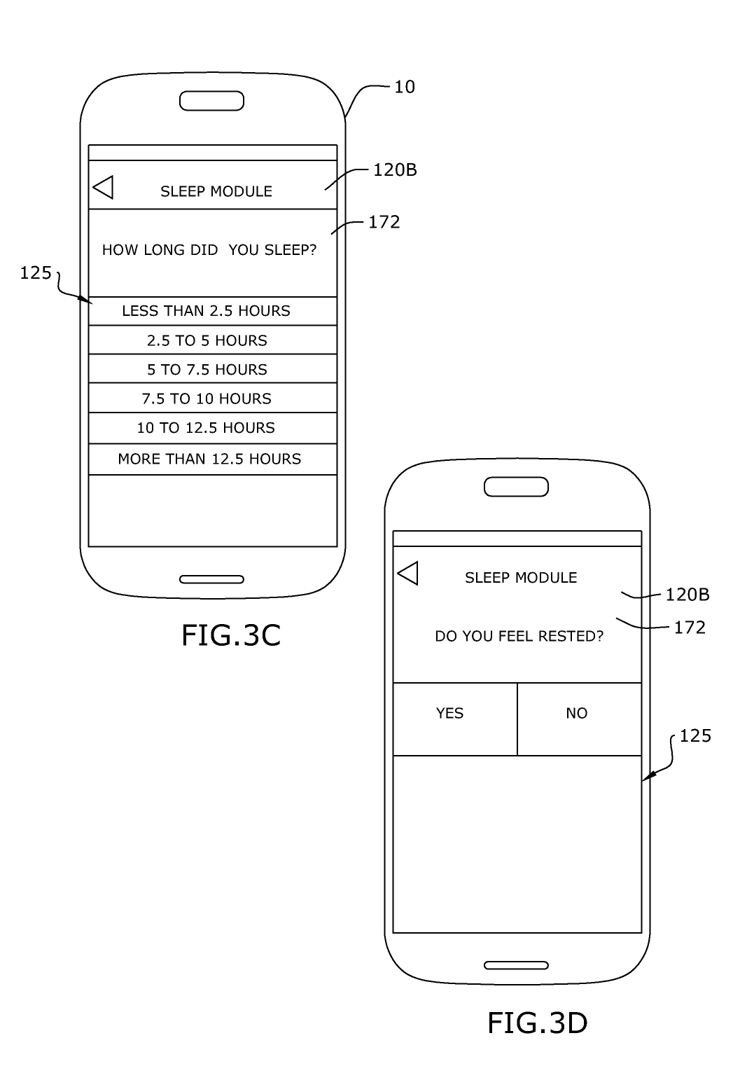

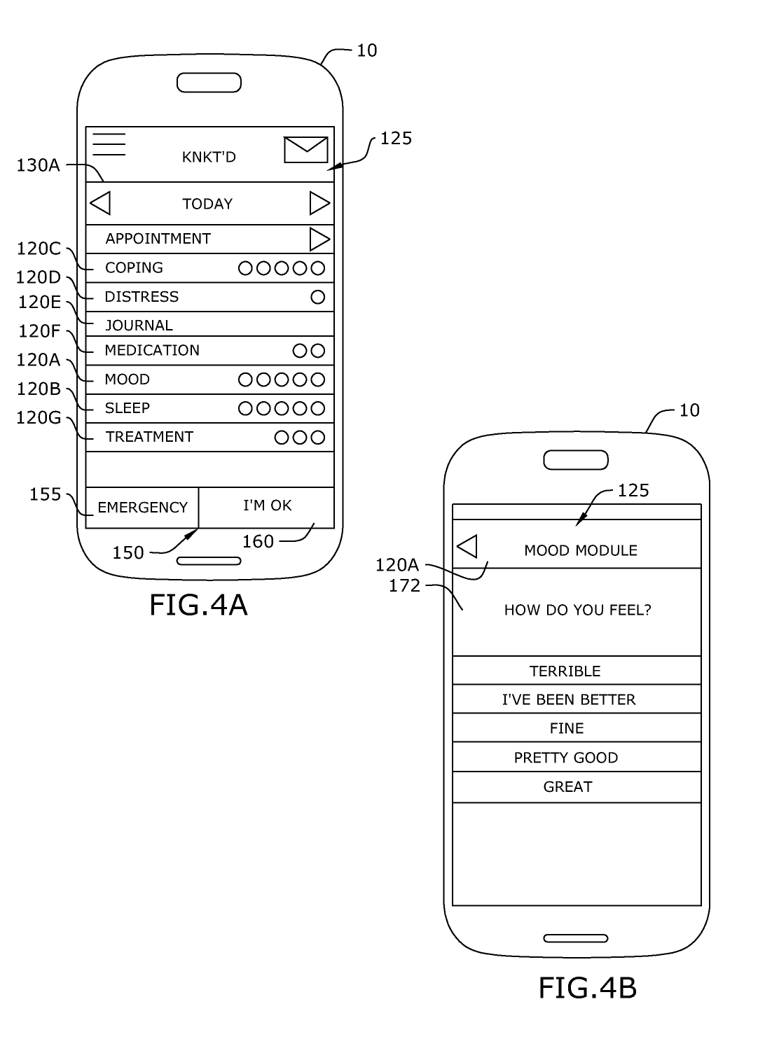

Referring now to FIGS. 3A-3D and 4A-4B, an exemplary embodiment of the subject technology is shown in the form of an electronic platform 125 accessible by an end user client 110 via a network connection. The platform 125 is shown in the form of a software application portal accessed via the computer system/server 10 in the form of an electronic mobile device. Referring to FIG. 3A, a user module interface dashboard 130 may include a plurality of user modules 120 available for selection. The modules 120 may pertain to the particular application the system is designed for. Each module 120 may be related to a different topic (for example, different behavioral wellness topics). The user module interface dashboard 130 may include a temporal aspect showing information for the day, week, or month. The user module interface dashboard 130 may include a messaging section 145 (shown with a welcome message however it will be understood that the section 145 may be used for direct communication between the end user and a party in the system). A status bar 150 may provide the end user with an option to immediately communicate whether they feel an emergency exists (via button 155) or feel no emergency is imminent (via button 160).

In an exemplary embodiment, the end user may select from a number of modules 120 assigned to his or her participation in the system. The exemplary embodiment may be helpful in monitoring the physical and behavioral health of a user by presenting for example a mood module 120A, a sleep module 120B, a coping module 120C, a distress module 120D, and a journal module 120E. While these particular modules 120 are shown, it will be understood that the modules for a particular user's account may vary. In an exemplary embodiment, the modules 120 displayed to the end user may be tailored to his or her program as directed by one or more providers assisting the end user. Each module 120 may include an indicator 140 (shown as radio buttons) which represent questions to be answered for the day. The responses to these questions may provide useful information for the day and for historical tracking. The information related to the responses may be used to generate real-time. In the example shown for behavioral wellness, real-time behavioral wellness reports may be generated. Aspects of the subject technology may trigger an alert to providers based on the content of the wellness reports. Alerts may be issued automatically or manually by a provider based on trends or responses that indicate an immediate need for attention. The indicators 140 may be white if the question has not been answered, greyed out or ghosted if the question is not yet posted or available for answering (allowing the provider to time-delay questions), or colored red, yellow, or green depending on the severity of the user's response. As may be appreciated, even at the dashboard stage, the user may be presented with a visual queue that his or health may be suffering. The visual display may be enough to alert the user to invoke the use of button 155 triggering a manual alert and plea for assistance. The status bar 155 may remain on screen when the end user interacts with any module 120 as seen below. In addition, an aspect of the subject technology may trigger an alert if the end user does not answer questions within a certain time frame (indicating potentially that the end user is avoiding others and potentially in a dangerous behavioral condition).

Referring now to FIG. 3B, a daily view for the sleep module 120B is shown when selected for example, from the screen shown in FIG. 3A. The sleep module 120B may include selectable questions (170; 172; 175) related to the user's sleep behavior. FIGS. 3C and 3D show screens invoked by selecting questions 172 and 175 in FIG. 3B. In the interfaces shown in FIGS. 3C and 3D, the user may respond to the questions 172; 175.

Referring now to FIGS. 4A and 4B, a user module interface dashboard 130a is shown according to another exemplary embodiment for a different end user than the end user's dashboard 130 shown in FIGS. 3A-3D. The dashboard 130a is the same as the dashboard 130 in FIG. 3A with the same modules 120A, 120B, 120C, 120D, and 120E (but ordered visually different from FIG. 3A) and also includes a medical module 120F and a treatment module 120G. Unlike the dashboard 130, the dashboard 130a does not necessarily include the status bar 150. FIG. 4B shows the result of the end user selecting the mood module 120A. The mood module 120A may include for example, survey question 172.

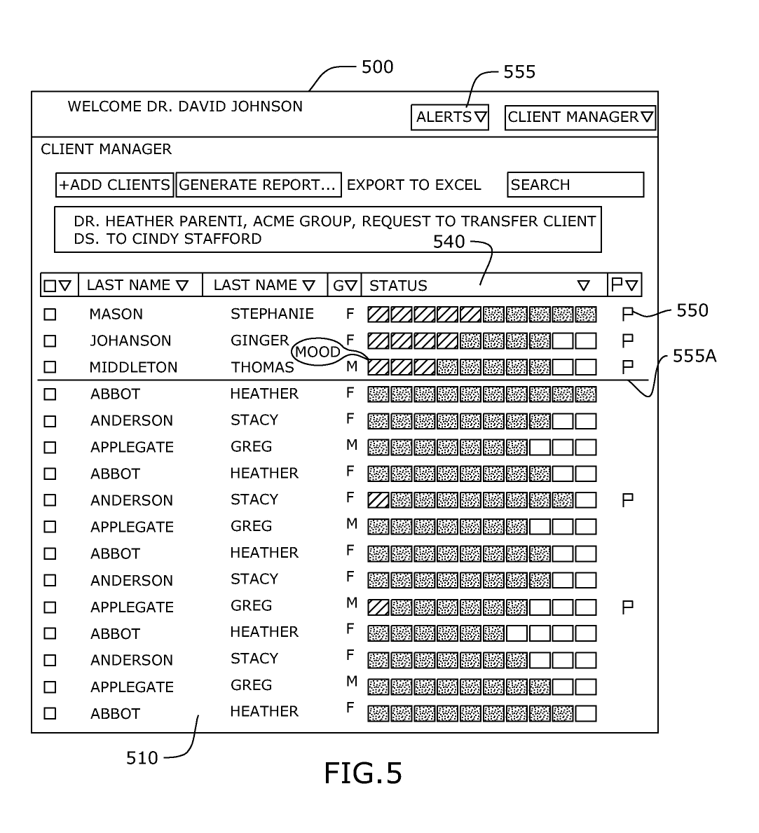

Referring now to FIG. 5, screenshot 500 of a client manager page 510 is shown according to an exemplary embodiment. The page 510 may be viewable from the provider 130 (FIG. 2) end of the system. The page 510 shows for example, a provider 130 (FIG. 2) a list of end users (clients) and their current health and/or behavioral status. The page 510 provides the provider 130 (FIG. 2) a convenient and readily visualized report of end user statuses. For example, each end user line may show a row of boxes or proxies representing the modules associated with the end user's program. The module represented by a box or proxy may be shown when selected (or if using a GUI, hovered over). Not all end users have all modules necessarily assigned to them. The boxes or proxies may be colored indicating a severity of the end user's physical or behavioral health for that module. In some embodiments, the boxes may be displayed in order of status (for example, red boxes may be listed first, then yellow, then green). Thus, the first box displayed for a user may represent a module that is different than the first box for a different end user (for example, the sleep module box for one user may be red and shown at the front of the row while the mood module box may be red and shown at the front of the row for another user). Blank boxes may just be placeholders. End users showing a red status in at least one module may be assigned a flag 550. A drop down alert box 555 may provide the function of highlighting end users requiring immediate attention and/or having initiated a manually triggered alert. End user's with for example, flags in at least three modules or end users which activated a crisis (emergency) button may be listed in the drop down alert box 555 for quick access throughout the system. A badge may show how many alerts are present in the drop down alert box 555. A status column 540 may be sortable so that end users with a red alert(s) in the system float to the top of the list and may be demarcated by a line 555A from the remaining end users. Below the demarcation line 555A, the end users may be listed according to a selected sort preference.

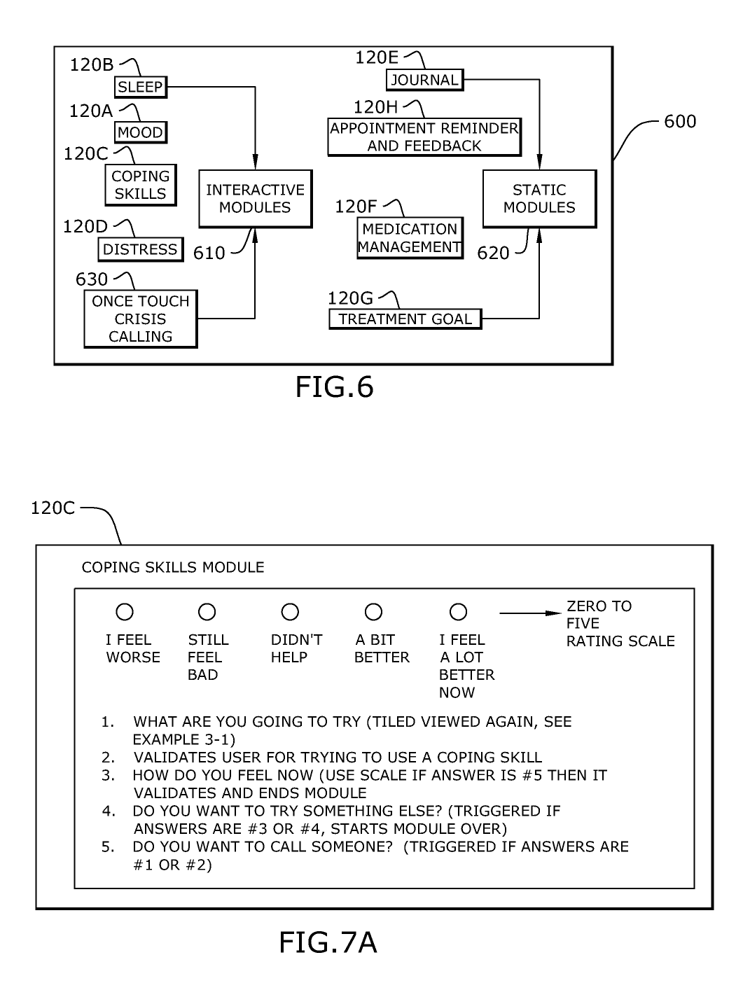

Referring now to FIG. 6, a chart 600 of some exemplary modules is shown. It will be understood that not all modules are shown and other modules may be contemplated depending on the application the system is designed for. Interactive modules 610 may be linked together so that the end user feedback/responses to one module may trigger the use of another module which may include the aforementioned mood module 120A, sleep module 120B, coping skills module 120C, distress module 120D, and a one-touch crisis calling module 630. The one-touch crisis calling module 630 may provide the end user with a function to establish an immediate alert and need for assistance. In an exemplary embodiment, the one-touch crisis calling module 630 may appear to the end user after end user input to questions is determined to demonstrate criteria that should trigger assistance. Static modules 620 may include modules that do not necessarily invoke another module but may be triggered as a result of selections within any of the interactive modules 610. The static modules 620 may include for example a journal module 120E, a medication management module 120F, a treatment goal module 120G, and an appointment reminder and feedback module 120H. The journal module 120E may provide a means for the user to record events, thoughts, or feelings that may be reviewed and monitored for trends. The medication management module 120F may provide a record of medication that is being taken, that needs to be taken, and that has or has not been taken when it should be. It may be appreciated that an alert may be triggered when the medication management module 120F indicates that medication has not been taken (for example, medication that regulates behavior). The treatment goal module 120G may track for example two goals that the user (or a provider) may easily update and monitor progress. The appointment reminder and feedback module 120H module may provide an interface to set appointments, remind the user of appointments, and provide feedback to providers about recent appointments attended. The appointment reminder and feedback module 120H may provide another form of data, for example, missed appointments that may trigger an alert.

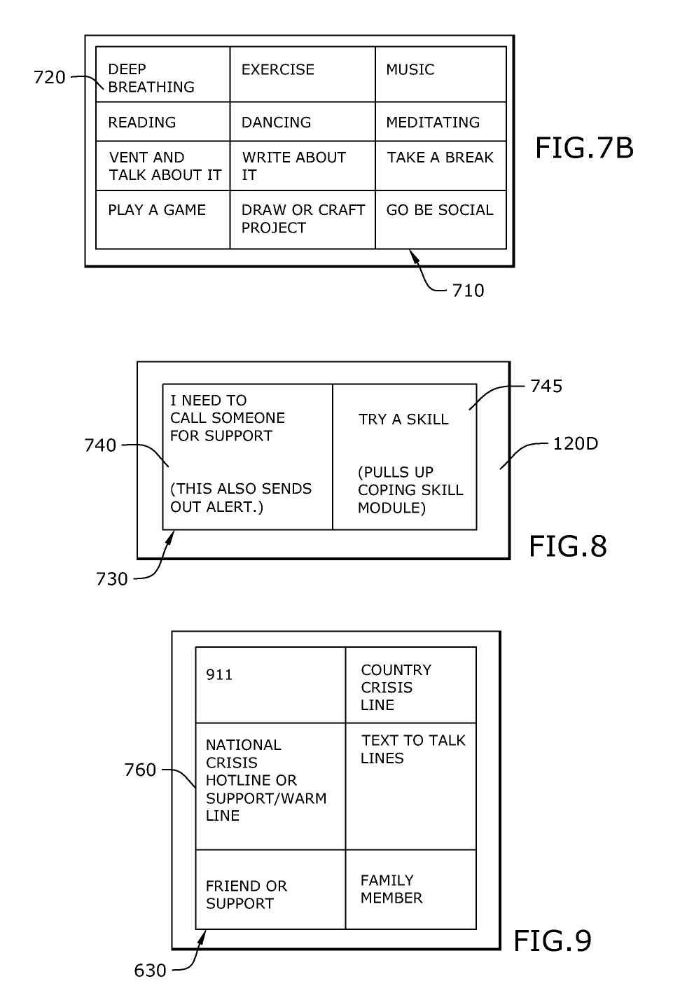

Referring now to FIG. 7A, a coping skills module 120C for monitoring behavioral health is shown according to an exemplary embodiment. The coping skills module 120C shows a rating scale for the end user to select how they are currently feeling. The coping skills module 120C also shows a list of follow-up module screens that may be triggered based on the selection in the rating scale. FIG. 7B shows a screen 710 of tiled suggested actions 720 in response to the end user selection in FIG. 7A.

Referring now to FIG. 8, a distress module 120 is shown according to an exemplary embodiment. The distress module 120D may include a tile view 730 including an option 740 to call someone for support (which may simultaneously trigger an alert and/or presentation of the crisis calling module 630) and an option 745 to try a skill. The option 745 to try a skill may trigger the system to present the end user with the coping skills module 120C. An exemplary embodiment of the crisis calling module 630 is shown in FIG. 9. The crisis calling module 630 may include a plurality of tiles 760 related to predetermined entities (including individuals personally known to the end user and to public assistance entities).

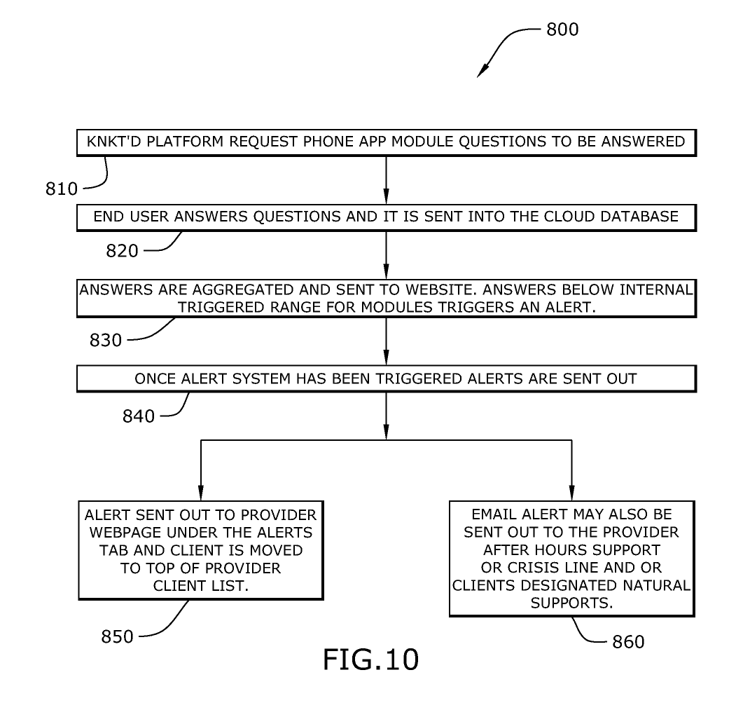

Referring now to FIG. 10, a method 800 of triggering an alert in the system is shown according to an exemplary embodiment. In block 810 a request to send module questions to an end user's device may be performed. The modules may be from more than one source depending on the end user's program and needs for monitoring. In block 820, responses from the end user may be transmitted to a database. In some embodiments, the database may be cloud based. In block 830, the responses may be aggregated and sent to a central portal for evaluation. Responses meeting predetermined criteria may trigger an alert. The criteria may be based on for example, trends in the end user's responses. For example, if a user has indicated a negative response to a question for more than 3 days in a row, an alert may be triggered. Of for example, if the responses to a question have not included a positive response for a predetermined number of days, an alert may be triggered. In block 840, in response to the system receiving an alert, the subject of the alert may be sent to providers monitoring the end user. In block 850, the alert may be sent to an electronic page a provider accesses for management of the end user. The alert may be given visual priority for the provider's attention. In block 860, an electronic message may be generated related to the alert which may be sent to the provider, an after-hours support or crisis line or to persons the end user has designated as support contacts.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

1. A computer program product for monitoring behavioral wellness of a monitored end user, the computer program product comprising a non-transitory computer readable storage medium having computer readable program code embodied therewith, the computer readable program code being configured to:
provide a portal, accessed through a computing device, for a monitored end user's input;
prompt the monitored end user on a display of the computing device for daily input of behavioral wellness data;
determine, via the processor, whether the monitored end user's daily input of behavioral wellness data are below a triggering range of wellness;
generate, via a processor, real-time behavioral wellness reports based on the monitored end user's daily input of behavioral wellness data;
trigger an alert for intervention to a medical provider based on the processor determining the monitored end user's daily input of behavioral wellness data is below the triggering range of wellness; and
provide the generated real-time behavioral wellness reports to the medical provider, the generated real-time wellness reports including the triggered alert for intervention.

2. The computer program product of claim 1, wherein the daily input of behavioral wellness data is in response to questions provided to the monitored end user through the portal.

3. The computer program product of claim 1, further comprising computer readable program code being configured to provide one or more module interfaces to the monitored end user, the one or more module interfaces related to a different behavioral wellness topic.

4. The computer program product of claim 3, further comprising computer readable program code being configured to provide a first module interface of a first behavioral wellness topic to the user and in response to the daily end user input of behavioral wellness data to the first module interface, invoking display of a second interface module of a second behavioral wellness topic.

5. The computer program product of claim 3, further comprising computer readable program code being configured to provide to the monitored end user recommended actions in response to the daily end user input of behavioral wellness data to one of the one or more module interfaces.

6. The computer program product of claim 3, wherein the one or more module interfaces are tailored to a program specific to the monitored end user.

7. The computer program product of claim 3, further comprising computer readable program code being configured to display indicators indicating a severity level of a monitored end user's wellness in relation to a question answered in the one or more module interfaces.

8. The computer program product of claim 1, further comprising computer readable program code being configured to provide the medical provider a prioritized display of a file related to the monitored end user in response to the alert for intervention being triggered.

9. The computer program product of claim 1, further comprising computer readable program code being configured to display on the computing device a push button notification configured for a user initiated plea for assistance.

10. The computer program product of claim 1, further comprising computer readable program code being configured to generate an electronic message to the medical provider indicating the alert for intervention has been triggered.
